(19) United States
(12) Patent Application Publication
(10) Pub. No.: US 2006/0167098 A1
(43) Pub. Date: Jul. 27, 2006

(54) Method for the treatment of diseases linked to an accumulation of triglycerides and cholesterol

(75) Inventor: Bernard Fromenty, Montmorency (FR)

Correspondence Address: OLIFF & BERRIDGE, PLC, P.O. BOX 19928, ALEXANDRIA, VA, 22320, US

(73) Assignee: Institut National De La Sante ET De LA Recherche Medicale (Inserm), Paris (FR)

(21) Appl. No.: 10/553,807

(22) PCT Filed: Apr. 15, 2004
(86) PCT No.: PCT/IB04/01166

§ 371 Date: Mar. 16, 2006

(30) Foreign Application Priority Data
Apr. 18, 2003 (IB) IB03/01463

(51) Int. Cl. A61K 31/22; A61K 31/195

(52) U.S. Cl. 514/561; 514/551

(57) ABSTRACT

The present invention concerns a method for the treatment and/or the prevention of diseases linked to the accumulation of triglycerides in tissues and blood comprising at least the step of administering to a human or non human animal in need thereof, as therapeutically active agent, an effective amount of (3-aminoisobutyric acid, derivative, prodrug, metabolite or complex thereof.

The invention relates to a method for the treatment and/or the prevention of diseases linked to an accumulation of triglycerides and cholesterol in tissues and blood of a human or non human animal.

Further, the invention relates to a method for the reduction of body fat mass in a human or non human animal having obese conditions or having a risk to manifest obese conditions.

Hyperlipemia and obesity afflict an increasing proportion of the population in western societies and may eventually lead to the clinical manifestations of coronary heart diseases, hepatic steatosis (i.e. fatty liver) and type 2 diabetes (i.e. non-insulin dependent diabetes mellitus; NIDDM).

Regarding obesity, it is a chronic disease that is associated with decreased life span and numerous medical problems. In particular, obesity-increases the risk of insulin resistance, type 2 diabetes, hepatic steatosis, hyperlipemia (including elevated levels of plasma triglycerides, cholesterol and free fatty acids), cholelithiasis, hypertension and other cardiovascular diseases.

Type 2 diabetes is an increasingly prevalent condition affecting approximately 150 million people worldwide. It is a heterogeneous and progressive condition resulting from impaired insulin action and secretion. In addition to its important role in the pathogenesis of a type 2 diabetes, insulin resistance, which is exacerbated by obesity, is also associated with increased cardiovascular risks and hypertension. It has also been associated with dyslipemia characterized by elevated triglycerides, quantitatively normal but small, dense low density lipoprotein (LDL) particles, and low high-density lipoprotein (HDL) cholesterol levels.

Accordingly, it is one object of the instant invention to provide a therapy for obesity that efficiently reduce or inhibit the gain of body fat mass induced by diets enriched in lipids and carbohydrates.

It is yet another object of the instant invention to prevent obesity, and once treatment has begun, to prevent progression or arrest the onset of diseases that are the consequences of, or secondary to, obesity, such as insulin resistance, type 2 diabetes, hyperlipemia, fatty liver, steatohepatitis and cardiovascular diseases.

Accordingly, it is another object of the instant invention to provide a therapy for type 2 diabetes that efficiently alleviates resistance or restores sensitivity to insulin.

Another object of the invention is to increase the blood level of adiponectin.

Adiponectin, which is secreted by adipocytes, is an insulin sensitizing hormone with blood concentration reduced in obesity and type 2 diabetes and insulin resistance. It has been observed that administration of recombinant adiponectin in rodents increased glucose uptake and fat oxidation in muscle, reduced hepatic glucose production in liver, and improved whole body insulin resistance. However, the exact receptors and signalling systems are unknown (Heilbronn et al., 2003, Curr Pharm Des, 9: 1411-8).

Otherwise, it is known that excess of fat in the body, including triglycerides and cholesterol in blood, linked or not to obesity condition, is a risk factor for cardiovascular diseases such as angina pectoris, myocardial infarction and hypertension. Hypertension can have a variety of uncomfortable and dangerous side effects and it is seen as a major risk factor in relation to coronary heart diseases. Specific ailments attributable to hypertension include heart failure, myocardial infarction, rupture or thrombus of the blood vessels in the brain, and kidney damages.

Accordingly, it is still another object of the invention to provide also a treatment for lowering the concentration of body fat, in particular lipids in the blood and/or as a preventive measure in people at risk due to high blood levels of cholesterol, and triglycerides.

Cholesterol and other lipids are transported through the blood stream in the form of round particles called lipoproteins. The two most commonly known lipoproteins implied in the transport of cholesterol are low-density lipoproteins (LDL) and high-density lipoproteins (HDL).

LDL-cholesterol is commonly called “bad” cholesterol. It can contribute to the formation of atherosclerosis plaques by deposing cholesterol onto the vessel wall. HDL-cholesterol is commonly called “good” cholesterol, and is a type of lipid that prevents the build-up of atherosclerosis plaques by favouring reverse cholesterol transport (i.e. the removal of cholesterol from peripheral tissues for delivery to the liver).

Therefore, it is of interest to balance the blood level of cholesterol in favour of HDL-cholesterol. This may be reached either by decreasing LDL over HDL-cholesterol (resulting in decrease of total amount of cholesterol) or decreasing LDL and HDL-cholesterol, but with a balance in favour of HDL-cholesterol (i.e. LDL decreases more than HDL). This may also be reached either by increasing HDL-cholesterol over LDL-cholesterol or increasing HDL and LDL-cholesterol, but with a balance in favour of HDL cholesterol, i.e. HDL increases more than LDL.

It is also an object of the present invention to provide a method for the treatment and/or the prevention of diseases linked to an imbalance of blood level of cholesterol in disfavour of HDL-cholesterol into a human or non human animal.

Thus, it has been observed that long term treatment with β-aminoisobutyric acid favours the balance of blood level of cholesterol toward HDL-cholesterol.

Accumulation of fat in the liver (i.e. hepatic steatosis, also called fatty liver) can be induced by various mechanisms such as excessive mobilization of fatty acids from adipose tissue, decreased hepatic fatty acid oxidation, increased fatty acid and triglyceride synthesis and decreased egress of lipoprotein from the liver (Fromenty B., Pessayre D., 1995 Pharmacol. Ther. 67:101-154). It is noteworthy that different mechanisms can coexist in a same individual.

Obesity, insulin resistance, type 2 diabetes and dyslipemia (including hyperlipemia) can induce macrovacuolar steatosis which is mainly an accumulation of triglycerides in the hepatocytes. In the long term (i.e. over, several years) steatosis can evolve toward steatohepatitis and cirrhosis. Steatohepatitis is characterized by a combination of steatosis (both macrovacuolar and microvesicular), necrosis (or apoptosis), inflammation and fibrosis. Steatohepatitis is a potentially severe liver disease that can lead to cirrhosis, liver failure, hepatocellular carcinoma, and death of the patient.

It has now been found that beta-aminoisobutyric acid (β-aminoisobutyric acid, also called “BAIBA”), presents beneficial effects on lipid homeostasis in obese (ob/ob) and lean (Swiss) mice.

In particular, in feeding experiments in lean mice treated with β-aminoisobutyric acid, in particular for 6 weeks, the results show that β-aminoisobutyric acid reduces the gain of body fat mass in Swiss mice fed with a standard diet or a western (hypercaloric) diet. In these experiments, β-aminoisobutyric acid significantly decreases the gain of body fat mass by 55% in mice fed with a standard diet and by 20% in mice fed with a hypercaloric diet. This makes β-aminoisobutyric acid a potent active agent for the treatment and/or the prevention of obesity.

Further, it has been shown that β-aminoisobutyric acid is efficient for decreasing liver triglycerides and reducing hypertriglyceridemia and hypercholesterolemia. With this respect, it has been first observed that β-aminoisobutyric acid increases mitochondrial beta-oxidation of fatty acids in liver. In particular, it has been shown that β-aminoisobutyric acid decreases liver and plasma triglycerides in mice fed with a hypercaloric diet and decreases liver triglycerides and plasma phospholipids and cholesterol in genetically obese ob/ob mice fed with a standard diet.

It has also been found that β-aminoisobutyric acid (BAIBA) presents a beneficial effect on insulin resistance in obese (ob/ob) mice.

In a set of feeding experiments in obese mice treated with β-aminoisobutyric acid for 6 weeks, the results showed that β-aminoisobutyric acid partly alleviate insulin resistance in obese mice fed with a standard diet. In those experiments, β-aminoisobutyric acid tended to decrease the glucose plasma level by around 20% and the insulin plasma level by around 35%. Furthermore, in other set of experiments, it has been shown that administration of β-aminoisobutyric acid in obese mice significantly increased plasma level of adiponectin by around 35%. Altogether, data obtained in ob/ob mice indicate that administration of β-aminoisobutyric tended to decrease plasma glucose and insulin and tended to increase plasma adiponectin after a 6-week period of treatment. It is noteworthy that in rodents and humans, increased plasma adiponectin is associated with increased insulin sensitivity (Berg A. H., Combs T. P. and Scherer P. E., 2002, Trends in Endocrinology & Metabolism 13: 84-89).

All these results are more precisely disclosed in the following experimental part.

Thus, β-aminoisobutyric acid appears to be particularly useful for the treatment of hyperlipidemic conditions and may be used as a preventive measure in people having risk due to high blood levels of cholesterol, and triglycerides and/or suffering from any type of disease linked to the accumulation of triglycerides and cholesterol in tissues and blood.

Accordingly, the present invention relates to a method for the treatment and/or the prevention of diseases linked to the accumulation of triglycerides and cholesterol, in tissues and blood comprising at least the step of administering to a human or non human animal in need thereof, as therapeutically active agent, an effective amount of β-aminoisobutyric acid, derivative, prodrug, metabolite or complex thereof.

More particularly, it relates to a method of treatment for lowering the blood levels of cholesterol, and/or triglycerides comprising at least the step of administering to a human or non human animal in need thereof, as therapeutically active agent, an effective amount of β-aminoisobutyric acid, derivative, prodrug, metabolite or complex thereof.

The present invention also relates to a method for the treatment and/or the prevention of diseases linked to the imbalance of cholesterol in disfavour of HDL-cholesterol in tissues and blood, comprising at least the step of administering to a human or non human animal in need thereof, as therapeutically active agent, an effective amount of β-aminoisobutyric acid, derivative, prodrug, metabolite or complex thereof.

In particular, it relates to a method of treatment for balancing the blood and/or tissues level of total cholesterol in favour of HDL-cholesterol comprising at least the step of administering to a human or non human animal in need thereof, as therapeutically active agent, an effective amount of β-aminoisobutyric acid, derivative, prodrug, metabolite or complex thereof.

The disease may be hyperlipemia (i.e. hypertriglyceridemia and/or hypercholesterolemia), hepatic steatosis, steatohepatitis and related liver diseases, insulin resistance, type 2 diabetes, syndrome X (i.e. metabolic syndrome), hypertension, angina pectoris and myocardial infarction. The term “metabolic syndrome” is inter alia characterized by hyperglycaemia, central obesity (i.e. accumulation of visceral fat), hepatic steatosis, dyslipemia and/or hypertension.

A further aspect of the invention relates to a method of treatment for the reduction or inhibition of the gain of body fat comprising at least the step of administering to human or non human animal in need thereof, as therapeutically active agent, an effective amount of β-aminoisobutyric acid, derivative, prodrug, metabolite or complex thereof.

Another aspect of the invention relates to a method of treatment for lowering liver level of triglycerides comprising at least the step of administering to a human or non human animal in need thereof, as therapeutically active agent, an effective amount of β-aminoisobutyric acid, derivative, prodrug, metabolite or complex thereof.

A further aspect of the invention relates to a method for the treatment or prevention of an obese condition, said method comprising at least the step of administering to a human or non human animal in need thereof, as therapeutically active agent, an effective amount of β-aminoisobutyric acid, derivative, prodrug, metabolite or complex thereof.

The present invention also related to a method for alleviating resistance or restoring sensitivity to insulin comprising at least the step of administering to a human or non human animal in need thereof, as therapeutically active agent, an effective amount of β-aminoisobutyric acid, derivative, prodrug, metabolite or complex thereof.

In particular, it relates to a method for the treatment or prevention of type 2 diabetes condition and related vascular diseases, said method comprising at least the step of administering to a human or a non human animal in need thereof, as therapeutically active agent, an effective amount of β-aminoisobutyric acid.

Another aspect of the invention relates to a method for lowering the blood levels of insulin and/or glucose comprising at least the step of administering to a human or non human animal in need thereof, as therapeutically active agent, an effective amount of β-aminoisobutyric acid, derivative, prodrug, metabolite or complex thereof.

In another aspect, the invention relates to a method of treatment for increasing the blood level of adiponectin comprising at least the step of administering to a human or non human animal in need thereof, as therapeutically active agent, an effective amount of β-aminoisobutyric acid, derivative, prodrug, metabolite or complex thereof.

β-Aminoisobutyric acid, is a natural β-amino acid generated during thymine and valine metabolism and is also metabolized in vivo mainly by the liver and gastrointestinal tissues in metabolites like methylmalonic acid semialdehyde, propionyl-coenzyme A, methylmalonyl-coenzyme A, and succinyl-coenzyme A (Griffith O. W., 1986, Annu. Rev. Biochem. 55: 855-878).

β-Aminoisobutyric acid may be used directly as active agent or may be generated in vivo after administration of a prodrug thereof like for example thymine or one of its intermediate metabolites.

The instant invention covers also the use of derivatives of β-aminoisobutyric acid.

The term “derivatives” include inorganic or organic salts, esters or amides of β-aminoisobutyric acid.

The terminal carboxylic group of β-aminoisobutyric acid may be in particular under the form of an ester, for example lower alkyl ester, (in particular in C₁-C₁₀) or of an amide.

More generally, its salts include not only the addition salts with carboxylic organic acids, like the acetate for example, but also other addition salts such as for example the trifluoroacetate, as well as the addition salts with inorganic acids such as the sulphate, hydrochloride and the like. The derivatives also include the salts resulting from the salivation of the carboxyl group and in particular the salts of alkali metals or alkaline earth metals such as the salts of sodium or of calcium.

In the scope of the present invention, the term “metabolite” is considered to be any substance resulting from the metabolism of BAIBA.

In the scope of the present invention, the term “prodrug” is dedicated to refer to any substance that gives rise to a pharmacologically active form of BAIBA although not itself active. However, it is particularly excluded from the definition of “prodrug” any peptide comprising BAIBA as amino-acid residue or pseudopeptide resulting from the coupling of BAIBA with non-peptidic entity, such as histamine.

In particular, the active agent is β-aminoisobutyric acid. It may be of L (i.e. S) or D (i.e. R) configuration or a mixture of L and D configurations.

The term “obesity” normally refers to a condition whereby an animal has an unusually elevated body fat mass resulting in an abnormally high Body Mass Index (BMI). As an example, adult humans are considered as obese if their BMI is above 30 kg/m².

In relation to obesity, the term “treatment” refers to a reduction of the severity of the disease, e.g. by reducing the body fat mass. The body fat mass refers to the total amount of lipids in an organism. These lipids include triglycerides, free fatty acids, cholesterol and cholesterol esters and phospholipids.

In relation to obesity, the term “prevention” refers to preventing obesity from occurring, i.e. β-aminoisobutyric acid is administered prior to the onset of the obese condition. This means that the compounds of the present invention can be used as prophylactic agents to impede an increase in body fat.

The terms “type 2 diabetes” normally refers to a chronic, lifelong disease that results when the body insulin does not work effectively. This disease is related to insulin resistance and is often accompanied by obesity and high cholesterol.

In relation to type 2 diabetes, the term “insulin resistance” refers to a defect in insulin stimulated glucose transport.

In relation to type 2 diabetes, the term “prevention” refers to preventing type 2 diabetes from occurring, i.e. β-aminoisobutyric acid is administered prior to the onset of the type 2 diabetes condition. This means that the compounds of the present invention can be used as prophylactic agents to impede a resistance to insulin.

The term “animals” includes mammals such as humans and farm (agricultural) animals, especially the animals of economic importance such as gallinaccous birds, bovine, ovine, caprine and porcine mammals, especially those that produce products suitable for the human consumption, such as meat, eggs and milk. Further, the term is intended to include fish and shellfish, such as salmon, cod, Tilapia, clams and oysters. The term also includes domestic animals such as dogs and cats. The term is also used to refer to laboratory animals which include, but are not limited to rodents such as mice, rats, guinea-pigs or hamsters.

In accordance with the methods indicated above, preferred embodiments are as follows: said animal is a human, an agricultural animal, a laboratory animal and/or a domestic or pet animal.

The treatment involves administering to an animal in need of such a treatment, a therapeutically effective amount of β-aminoisobutyric acid in the blood of the animal for the duration of the period of its administration.

A further aspect of the invention relates to the use of β-aminoisobutyric acid, derivative, prodrug, metabolite or complex thereof, as therapeutically effective agent for the preparation of a pharmaceutical composition for the treatment and/or prevention of diseases linked to an accumulation of triglycerides and/or cholesterol in tissues and blood.

A further aspect of the invention relates to the use of β-aminoisobutyric acid, derivative, prodrug, metabolite or complex thereof, as therapeutically effective agent for the preparation of a pharmaceutical composition for the treatment and/or prevention of diseases linked to an imbalance of blood and/or tissues level of cholesterol in disfavour of HDL-cholesterol.

A further aspect of the invention relates to the use of β-aminoisobutyric acid, derivative, prodrug, metabolite or complex thereof, as therapeutically effective agent, for the preparation of pharmaceutical composition intended for the treatment and/or prevention of diseases linked to an accumulation of insulin and/or glucose and/or to a decrease of adiponectin.

Another aspect of the invention relates to the use of β-aminoisobutyric acid, derivative, prodrug, metabolite or complex thereof, as therapeutically effective agent, for the preparation of a pharmaceutical composition intended for alleviating the resistance or restoring the sensibility to insulin.

A further aspect of the invention relates to the use of β-aminoisobutyric acid, derivative, prodrug, metabolite or complex thereof, as therapeutically effective agent, for the preparation of a pharmaceutical composition intended for the treatment and/or the prevention of type 2 diabetes.

The pharmaceutical composition according to the present invention is more particularly directed to the treatment and/or the prevention of hyperlipemia (i.e. hypertriglyceridemia and/or hypercholesterolemia), hepatic steatosis, steatohepatitis, diabetes, metabolic syndrome (X syndrome), hypertension, angina pectoris and myocardial infarction.

A further aspect of the invention relates to a pharmaceutical composition, in particular useful for the prevention and/or treatment of diseases linked to an accumulation of cholesterol, and triglycerides in tissues and blood, comprising at least as therapeutically active agent, an effective amount of β-aminoisobutyric acid, derivative, prodrug, metabolite or complex thereof.

A further aspect of the invention relates to a pharmaceutical composition, in particular useful for the prevention and/or treatment of diseases linked to an imbalance of blood and/or tissues level of cholesterol in disfavour of HDL-cholesterol.

The pharmaceutical composition according to the invention is more particularly useful for the treatment or prevention of hypertension, fatty liver, metabolic syndrome and an obese condition.

The pharmaceutical composition according to the invention is more particularly useful for the treatment or prevention of type 2 diabetes.

A further aspect of the invention relates to a pharmaceutical composition in particular useful for alleviating the resistance or restoring the sensitivity to insulin.

Another aspect of the invention relates to a pharmaceutical composition particularly useful for lowering blood level of insulin and/or glucose.

Another aspect of the invention relates to a pharmaceutical composition particularly useful for increasing blood level of adiponectin.

Preferably, the pharmaceutical composition comprises in admixture with β-aminoisobutyric acid a pharmaceutically acceptable carrier or excipient.

The invention also relates to a nutritional composition comprising as a nutritional active agent an amount of β-aminoisobutyric acid, derivative, prodrug, metabolite or complex thereof effective to reduce, or to prevent, an increase in the total body fat mass in human or non-human animal, and a method for producing reduction of the fat mass in a, human or non-human animal in need thereof, comprising administering thereto an effective amount of said nutritional composition.

Another aspect of the present invention relates to a nutritional composition comprising as a nutritional active agent an amount of β-aminoisobutyric acid, derivative, prodrug, metabolite or complex thereof effective to alleviate resistance or restore sensibility to insulin, and a method for alleviating resistance or restoring sensibility to insulin in a human or non-human animal in need thereof, comprising administering thereto an effective amount of said nutritional composition.

Another aspect of the present invention relates to a nutritional composition comprising as a nutritional active agent an amount of β-aminoisobutyric acid, derivative, prodrug, metabolite or complex thereof effective to balance blood and/or tissue level of cholesterol in favour of HDL-cholesterol, and a method for balancing blood and/or tissue cholesterol in favour of HDL-cholesterol in a human or non-human animal in need thereof, comprising administering thereto an effective amount of said nutritional composition.

The nutritional composition may be any food composition. In particular, it may be a drink or a powder that can be reconstituted to produce such a drink. It may include other nutritional components like vitamins, stabilisers, antioxidants, emulsifiers, flavouring agents.

Preferred embodiments relate to a condition wherein the animal has developed an obese condition or is low energy adapted.

The term “low energy adapted” refers to a condition whereby an animal has a low energy consumption, i.e. less than normal.

As a pharmaceutical medicament, the compounds of the present invention may be administered directly to the animal by any suitable technique, including parenterally, intranasally, orally, or by absorption through the skin. They can be administered locally or systemically. The specific route of administration of each agent will depend, e.g., on the medical history of the animal.

Examples of parenteral administration include subcutaneous, intramuscular, intravenous, intraarterial, and intraperitoneal administration.

The term “effective amount” means the minimal amount necessary to observe the expected effect i.e. a lowering effect on the concentration of triglycerides and/or cholesterol in tissues and blood, and/or a balancing effect of blood level of cholesterol in favour of HDL-cholesterol; and/or reduction of insulin and/or glucose in blood; and/or increase of adiponectin in blood.

In particular, it might be in the range of about 5 mg/kg/day to 1000 mg/kg/day of patient body weight, in particular about 50 mg/kg/day to 500 mg/kg/day.

Generally, the formulations are prepared by contacting the compounds of the present invention each uniformly and intimately with liquid carriers or finely divided solid carriers or both.

In addition, the compounds of the present invention may be appropriately administered in combination with other treatments for combating or preventing the diseases considered according to the invention and/or the obesity.

The invention will be more fully understand by reference to the following examples. They should not, however, be constituted by limiting the scope of the invention.

### LEGENDS OF THE FIGURES

FIG. 1: it shows effects of β-aminoisobutyric acid (BAIBA) on body fat mass in Swiss mice fed with a standard diet, with or without BAIBA in the drinking water, and fasted for 48 hours before DEXA measurements (i.e. 2 and 6 weeks after the initiation of the treatment). Results are expressed as the percentage of the control values. Asterisk (*) indicates a significant difference (p<0.01) between the groups.

FIG. 2: it shows effects of β-aminoisobutyric acid (BAIBA) on the gain of body fat mass (as assessed by DEXA) in Swiss mice fed with a standard diet with or without BAIBA in the drinking water (8 mice in each group). Variations observed for the whole period of the investigation (T0-Tsix weeks) are also shown. Results are expressed in gram. Asterisk (*) indicates a significant difference (p<0.01) between the groups.

FIG. 3: it shows effects of β-aminoisobutyric acid (BAIBA) on the gain of body fat mass (as assessed by DEXA) in Swiss mice fed with a western diet (WD) with or without BAIBA in the drinking water (8 mice in each group). For comparison, the experiment included a group of mice (n=8) fed with a standard diet (SD). Variations observed for the entire period of the investigation (T0-Tsix weeks) are also shown. Results are expressed in gram. Asterisk (*) indicates a significant difference (p<0.01) between Control-WD and BAIBA-WD mice.

FIG. 4: it shows effects of β-aminoisobutyric acid (BAIBA) on liver lipids in Swiss mice fed with a western diet (WD) with or without BAIBA in the drinling water. For comparison, the experiment included a group of mice fed with a standard diet (SD). Mice in this experiment are those studied in FIG. 3 but after the last DEXA measurement (i.e. after 6 weeks) animals were fasted for 48 hours and killed for hepatic lipid determination. Total lipids (mg/whole liver) and triglycerides (mg/whole liver) were determined in 7, 8 and 8 mice, respectively in Controls-SD, Controls-WD and BAIBA-WD mice. Asterisk (*) indicates a significant difference (p<0.05) between Control-WD and BAIBA-WD mice.

FIG. 5: it shows effects of β-aminoisobutyric acid (BAIBA) on liver triglycerides in obese (ob/ob) mice were fed with a standard diet, with (10 mice) or without (7 mice) BAIBA in the drinking water. After 6 weeks of investigation, animals were fasted for 48 hours and killed for hepatic lipid determination. The results are expressed as mg/whole liver and mg/gram of lipids. Asterisk (*) indicates a significant difference (p<0.01) between control mice and mice receiving BAIBA.

FIG. 6: it shows effects of β-aminoisobutyric acid (BAIBA) on plasma lipids in Swiss mice fed with a western diet (WD) with or without BAIBA in the drinking water. For comparison, the experiment included a group of mice fed with a standard diet (SD). Mice in this experiment are those studied in FIGS. 3 and 4 (i.e. after 6 weeks of treatment and the last DEXA measurement blood was collected in fasted mice before killing for liver lipid determination). Plasma triglycerides (TG), phospholipids (Ph.L), total cholesterol (Chol) and non-esterified fatty acids (NEFA) were determined in 8 mice for each group (Controls-SD, Controls-WD and BAIBA-WD).

FIG. 7: it shows effects of β-aminoisobutyric acid (BAIBA) on plasma lipids in genetically obese (ob/ob) mice fed with a standard diet, with (10 mice) or without (7 mice) BAIBA in the drinking water. After 6 weeks, mice were fasted for 48 hours and blood was collected for the determination of plasma triglycerides (TG), phospholipids (Ph.L) and total cholesterol (Chol). Mice in this experiment are those studied in FIG. 5. Asterisk (*) indicates a significant difference (p<0.05) between control ob/ob mice and ob/ob mice receiving BAIBA.

### MATERIALS AND METHODS

Six to 8 weeks old male (Crl:CD-1(ICR)BR) Swiss mice, and six to ten weeks old male RjOrl Swiss mice (i.e. lean mice) weighing 28 and 32 grams were purchased from Dépré (Saint Doulchard, France). Ten to 12 weeks old ob/ob male (C57BL/6-ob) mice (i.e. obese mice), weighing 40 to 44 grams were purchased from Janvier (Le-Genest-St-Isle, France). In one experiment, younger ob/ob mice (5-6 weeks old) weighing 25-26 grams were also studied. In genetically obese ob/ob mice, a mutation on the gene of leptin prevents the normal production of this hormone, thus increasing appetite and decreasing energy consumption (Friedman J. M., Halaas J. L., 1998, Nature 395 : 763-770). Accordingly, ob/ob mice exhibit severe (i.e. “morbid”) obesity, massive steatosis, insulin resistance and diabetes (Friedman and Halaas, 1998 see previously; Koteish A., Diehl A. M., 2001, Semin. Liver Dis. 21: 89-104). The animals were acclimatized for one to two weeks before the start of experiments.

In our investigations, two kinds of diet were used: 1—a standard diet (A04 diet from UAR) containing 3% of lipids and bringing 2900 kcal per kg; 2—a western diet (from UAR too) containing 16% of lipids and bringing 4300 kcal per kg.

D,L-β-Aminoisobutyric acid (BAIBA) was purchased from Sigma-Aldrich. BAIBA has been administered in drinking water at the dose of 100 mg/kg/day or 500 mg/kg/day for 6 weeks, 2 months or 4 months according to the experiments.

The in vivo determination of body fat mass in anesthetized mice has been performed by “DEXA” (Dual Energy X-ray Absorptiometry) (Pietriobelli A., Formica C., Wang Z., Heymsfield S. B., 1996, Am. J. Physiol. 271 (Endocrinol. Metab. 34): E941-E951)). Hence, in our study, the body fat mass is the total amount of lipids (in grams) per animal (excluding its head) that is quantified by DEXA. It is noteworthy that DEXA also gives for each investigated animal its lean mass that is the total amount of water and proteins (in grams) in the body (excluding the head). Therefore, for a given animal, DEXA allows the determination of the percentage of body fat which is its body fat mass divided by the sum of its body lean and fat masses (fat mass/(lean mass+fat mass)). The “DEXA” apparatus was a Piximus® from Lunar Corporation (Madison, Wis.). Mice were anesthetized thanks to a mixture of xylazine and ketamine.

In this study, two types of procedures were used, according to the nutritional state of animals at the moment of “DEXA” measurements:

Procedure 1: the animals were not fasted before DEXA investigations. The first DEXA measurement was performed one day before the beginning of the investigation (T0). Afterwards, DEXA measurements have been respectively performed two weeks (T2) and six weeks (T6) after the beginning of the treatment. With this procedure, it has been possible to determine in each group of animals the evolution of several parameters (e.g. fat mass, body weight) between respectively T0 and T2, and T2 and T6. Comparisons of these evolutions were thus performed for the T0-T2, T2-T6 and T0-T6 periods.

Procedure 2: the animals were fasted for 48 hours before DEXA measurements which have been performed 2 weeks (T2) and 6 weeks (T6) after starting the treatment. In this procedure, DEXA measurement before the beginning of treatment has not been performed. Comparisons of the different parameters (e.g. fat mass, body weight) between both groups were performed at T2 and at T6.

Total lipids and triglycerides in the liver of animals were assessed according to a procedure partially reported by (Letteron P., Fromenty B., Terris B., Degott C., Pessayre D., 1996, J. Hepatol. 24 : 200-208). Briefly, after killing of the animals, livers were removed and homogenized in sterile water. Hepatic lipids were thus extracted by a mixture of chloroform and methanol (2/1; v/v). After removal of the aqueous phase, the organic phase (chloroform containing the lipids) was evaporated and the amount of lipids was determined by gravimetry. Lipids were subsequently resuspended in isopropanol (final concentration, ca. 10 mg/mL). After removal of the phopholipids by using aluminum hydroxide hydrate, triglycerides were determined colorimetrically by using periodate and the Nash's reagent (containing acetylacetone, ammonium acetate and isopropanol). The reaction generates diacetyldihydrolutidine which is assayed by spectrophotometry (λ=410 nm).

Plasma lipids (triglycerides, total cholesterol, phospholipids) were measured on an automatic analyser (Hitachi 717®). The commercial kits used to assess triglycerides, total cholesterol and phospholipids on this analyser were all from bioMérieux (references 61238, 61219 and 61491, respectively). Plasma non esterified fatty acids (NEFA) were assessed by using a commercial kit (Wako, reference 994-75409). Hepatic and plasma lipids were performed at the end of six weeks of treatment in animals that have been fasted for 48 hours. Triglycerides and HDL-cholesterol were also assessed after 2 months (in the fed state) or after 4 months of treatment (after an overnight fast).

Plasma HDL-cholesterol and alanine aminotransferase (ALT) were measured on an automatic analyser (Olympus AU400®) with commercial kits (Olympus Diagnostica GmbH, references OSR6187 and OSR6107, respectively).

Plasma glucose was measured on an automatic analyser (Synchron LX20 from Beckman Coulter) with a commercial kit (kit n°472500 from Beckman).

Plasma insulin, adiponectin and leptin were measured by radioimmunoassay (RIA). Insulin was measured with the Insulin-CT kit from Cis Bio International (Gif-sur-Yvette, France), whereas plasma leptin and adiponectin were assessed with kits from Linco Research Inc. (St Charles, Mo.). Leptin was not measured in ob/ob mice since these animals are genetically leptin-deficient (Friedman J. M., Halaas J. L., 1998, Nature 395: 763-770). Results regarding insulin were expressed as recommended by kit manufacturer.

Finally, liver histology was performed according to regular procedure. Steatosis and fibrosis were assessed thanks to the Oil red O and Masson's trichrome stains, respectively, whereas necrosis and inflammation were determined thanks to the HPS (Hematoxylin-Phloxin-Saffron) stain, or the HE (Hematoxylin-Eosin) stain. In one experiment, perisinusoidal fibrosis was assessed with the Sirius Red stain.

The results are expressed as mean±SEM (standard error of the mean). A t test of Student was used to look for a statistic difference between values obtained for the control group and the group of mice treated with BAIBA. This difference was considered as statically significant for a value of p<0.05. The numbers indicated between the parentheses represent the numbers of animals used in the experiments.

### EXAMPLE 1

### Beneficial Effects of BAIBA on Body Fat

a) Effect of BAIBA in Lean Mice After a Fasting Period

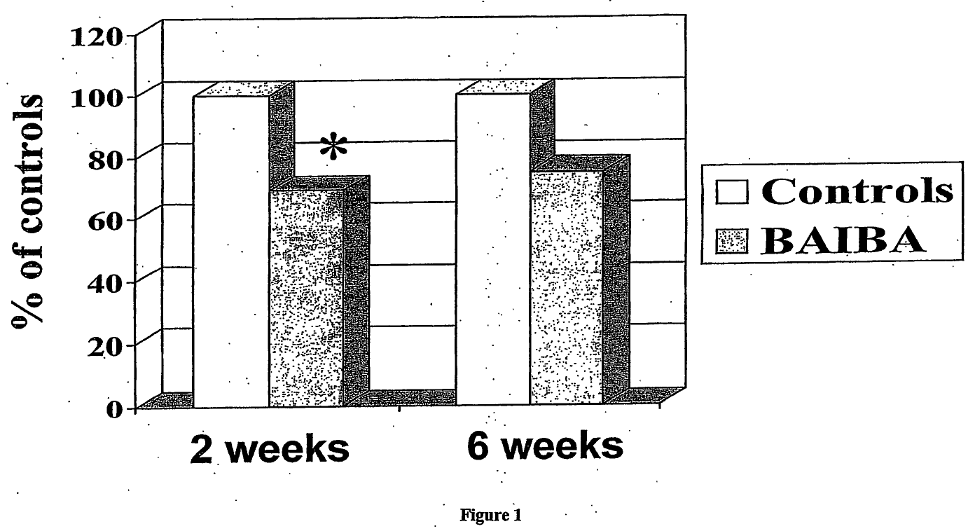

In a first series of experiments, the effects of 100 mg/kg/day of β-aminoisobutyric acid (BAIBA) on body fat were investigated in Swiss (lean) mice fed with a standard diet (A04 chow) and fasted for 48 hours before DEXA measurements. Mice were 6 to 8 weeks old at the beginning of the experiment. DEXA was performed twice after the initiation of the treatment. In this experiment, BAIBA decreased body fat mass by 31% and 25%, respectively after 2 and 6 weeks of treatment. The results in FIG. 1 indicate that:
after 2 weeks, body fat mass was 2.63±0.23 and 1.80±0.11 grams, respectively in control mice (n=7) and mice receiving BAIBA (n=7);
after 6 weeks, body fat mass was 3.40±0.27 and 2.56±0.32 grams, respectively in control mice and mice receiving BAIBA.

In this experiment, body weight was not significantly different between control mice and mice receiving BAIBA, after 2 and 6 weeks of treatment (data not shown).

b)—Effect of BAIBA in Lean Mice Not Submitted to Fast

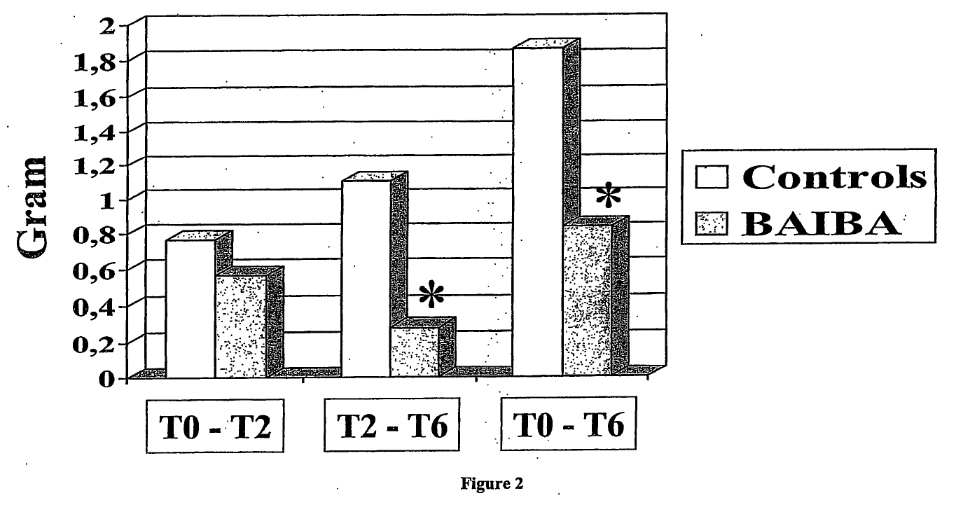

In a second series of investigations, we sought to determine as to whether of 100 mg/kg/day of β-aminoisobutyric acid (BAIBA) decreases body fat in Swiss mice not submitted to fast before DEXA measurements. Mice were 6 to 8 weeks old at the beginning of the experiment. In this experiment, DEXA was performed one day before the beginning of the treatment (T0) and then two and six weeks after the initiation of the experience (T2 and T6, respectively). This allowed us to determine for each animal the variation of body fat mass for the different periods of the treatment (T0-T2, T2-T6 and T0-T6). In this experiment, we found that BAIBA reduced the gain of body fat mass in Swiss mice and that this effect was more pronounced in the second period (T2-T6) of the treatment. This effect of BAIBA is submitted in FIG. 2. It shows that:
through the T0-T2 period, control mice (n=8) gained 0.76±0.06 grams of body fat whereas mice receiving BAIBA (n=8) gained 0.56±0.12 grams.
through the T2-T6 period, control mice gained 1.10±0.07 grams of body fat whereas mice receiving BAIBA gained 0.28±0.12 grams.
Overall, for the entire period of the experiment (T0-T6), BAIBA reduced the gain of fat mass by 55%, as control mice and mice receiving BAIBA gained respectively 1.86±0.11 and 0.84±0.18 grams of body fat.

In this experiment, BAIBA slowed the gain of body weight during the 6-week period of treatment. Indeed, through the T0-T6 period, BAIBA significantly (p<0.01) reduced the gain of body weight by 12%, as control mice and mice receiving BAIBA gained respectively 10.30±0.34 and 9.02±0.42 grams.

c)—Effect of BAIBA in Lean Mice Fed with a Western Diet

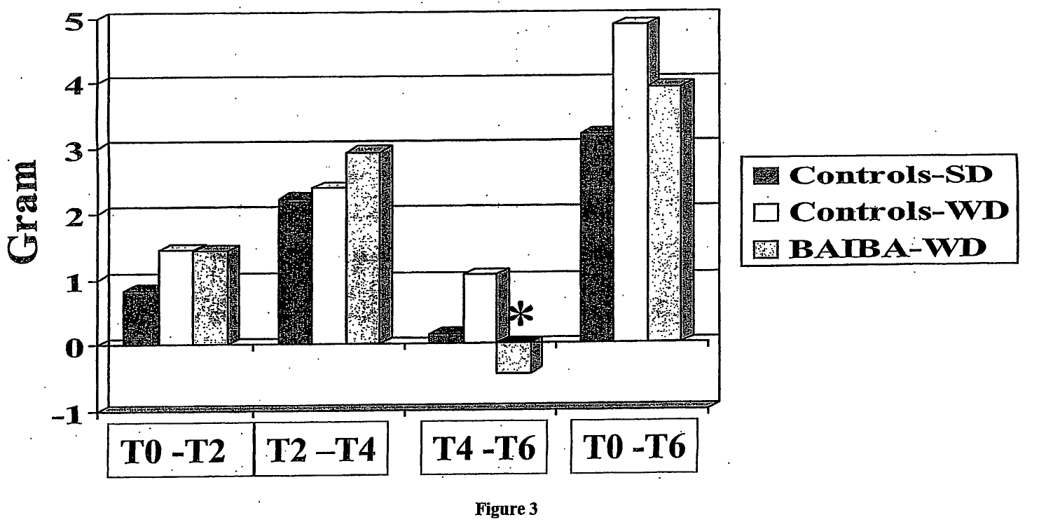

In a third series of experiments, we sought to determine as to whether of 100 mg/kg/day of β-aminoisobutyric acid (BAIBA) would be able to decrease body fat mass in Swiss mice fed with a western diet (i.e. hypercaloric diet). For this purpose, young (4 weeks old at T0) Swiss mice were fed with a hypercaloric western diet (WD), with or without BAIBA in the drinking water (8 mice in each group). For comparison, the experiment included a group of mice (n=8) fed with a standard diet (SD). DEXA was performed one day before the beginning of the treatment (T0) and then two, four and six weeks after the initiation of the experience (T2, T4 and T6, respectively). This allowed us to determine for each animal the variation of body parameters (e.g. fat mass, body weight) for the different periods of the treatment (T0-T2, T2-T4, T4-T6 and T0-T6). For these four DEXA determinations (T0 to T6), mice were not fasted before measurements. In this experiment, BAIBA reduced the gain of body fat mass in Swiss mice fed with a western diet and this beneficial effect was observed from the fourth week of the BAIBA administration. This effect is illustrated in FIG. 3. It shows that:
during the T4-T6 period control mice fed with the western diet gained 1.06±0.34 grams of body fat whereas mice fed with the same diet and receiving BAIBA lost 0.45±0.30 grams of body fat (significantly different, p<0.05),
through the entire period of the treatment (T0-T6), the gain of body fat mass was reduced by 20% in mice fed with the western diet and receiving BAIBA. Indeed, during the T0-T6 period control mice fed with the western diet (Controls-WD) gained 4.88±0.83 grams of body fat whereas mice fed with the same diet and receiving BAIBA (BAIBA-WD) gained 3.91±0.59 grams of body fat mass. For comparison, mice fed with the standard diet (Controls-SD) gained 3.18±0.34 grams of body fat during the entire period (T0-T6) of the experiment.
At the end of the treatment (T6), the body fat mass was 4.48±0.33, 6.21±0.85 and 5.25±0.60 grams, respectively for the Controls-SD, Controls-WD and BAIBA-WD groups of mice (15% decrease between Controls-WD and BAIBA-WD). The percentage of body fat ((fat mass)/(fat+lean mass)) was at this time 13.23±0.86, 17.96±2.04 et 15.76±1.53%, respectively in the Controls-SD, Controls-WD and BAIBA-WD groups. Altogether, these results indicate that the western diet increased body fat mass in Swiss mice over a 6-week period and that BAIBA was able to curb this abnormal gain of body fat.

In this experiment, BAIBA tended to reduce body weight in mice fed with the western diet. Indeed, after the 6-week period of treatment body weight was 37.9±1.14 and 37.0±0.76 grams, respectively in control mice (Controls-WD) and mice receiving BAIBA (BAIBA-WD).

Finally, we sought to determine on the very same animals as to whether fasting would be able to enhance the beneficial effect of BAIBA on body fat mass. Thus, after the fourth DEXA measurement (i.e. DEXA determination at T6), mice were allowed to recover from the anaesthesia for ca. 2 days and were then submitted to a 48-hour period of fast. A last DEXA measurement was then performed in fasted mice. Our results showed that fasting did not increase the beneficial effect of BAIBA. Indeed, after the fasting period, body fat mass was 3.10±0.30, 5.40±0.75 et 4.79±0.56 grams, respectively in control mice fed the standard diet (Controls-SD), control mice fed with the western diet (Controls-WD), and mice fed with the western diet and receiving BAIBA (BAIBA-WD). Thus, in the animals fed with the western diet for 6 weeks, BAIBA decreased by 15 and 11% the percentage of body fat (determined at T6 or a few days later), respectively in the fed and the fasted state. These results indicate that a reduction of food intake does not provide further benefit to the favourable effect of BAIBA on body fat. At the end of the fasting period, body weight was 32.0±0.6, 34.2±1.1 and 33.6±0.9 grams, respectively in the Controls-SD (n=8), Controls-WD (n=8), and BAIBA-WD (n=8) groups of mice.

d)—Effect of BAIBA in Genetically Obese ob/ob Mice:

In a last series of investigations, we sought to determine as to whether of 100 mg/kg/day of β-aminoisobutyric acid (BAIBA) would be able to decrease body fat mass in genetically obese ob/ob mice. In these investigations 6 weeks and 10-12 weeks old ob/ob mice were fed with a standard diet, and DEXA measurements were performed one day before the beginning of the treatment (T0) and then two and six weeks after the initiation of the experience (T2 and T6, respectively). DEXA was performed on fed mice. Our results showed that BAIBA presented a slight beneficial effect on body fat only in the older animals (10-12 weeks old). Indeed, in these mice BAIBA afforded an 11% reduction of body fat in the first two weeks of treatment, since control (n=6) and treated (n=6) mice respectively gained 5.20±0.63 and 4.65±0.35 grams (not significantly different). This beneficial effect of BAIBA was no longer observed during the T2-T6 period, and during the whole T0-T6 period, control and treated mice respectively gained 7.43±0.51 and 7.30±0.61 grams of body fat. It is noteworthy that despite the limited effect of BAIBA on body fat mass in ob/ob mice at the end of the 6-week period of treatment, further investigations show that BAIBA afforded a proportional stronger reduction of liver triglycerides and plasma cholesterol after 6 weeks of investigation (see below).

### EXAMPLE II

### Beneficial Effects of BAIBA on Liver Lipids

a)—Effect of BAIBA in Lean Mice Fed with a Western Diet:

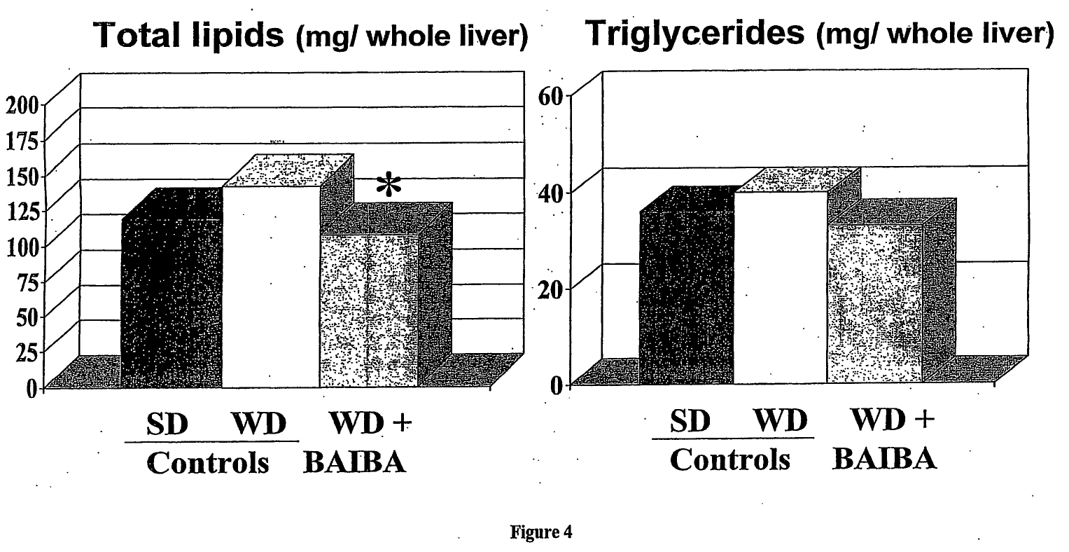

Liver lipids were assessed in mice fed with the western diet (WD) receiving or not of 100 mg/kg/day of β-aminoisobutyric acid (BAIBA) in the drinking water (8 mice in each group). For comparison, the experiment also included a group of mice (n=8) fed with a standard diet (SD). These animals were those used for the determination of body fat by DEXA (see above, paragraph I-c) but after the last DEXA measurement (i.e. after 6 weeks of treatment), mice were allowed to recover from the anaesthesia for ca. 2 days and were then submitted to a 48-hour period of fast. At the end of this fasting period DEXA was again performed (see above) and then mice were killed for liver lipids determination. The western diet was responsible for an increase in liver lipids as compared to mice fed with the standard diet. In the animals fed with the western diet, BAIBA was found to decrease total lipids and triglycerides, respectively by 24 and 17%. This effect is illustrated in FIG. 4 which shows that:
liver lipids were 119±16, 142±13 and 108±7 mg/whole liver, respectively in control mice fed the standard diet (Controls-SD, 7 mice), control mice fed with the western diet (Controls-WD, 8 mice), and mice fed with the western diet and receiving BAIBA (BAIBA-WD, 8 mice);
hepatic triglycerides were 36±10, 40±8 and 33±6 mg/whole liver, respectively in the Controls-SD, Controls-WD and BAIBA-WD groups. Taken together, these results indicate that although the western diet increased liver lipids and triglycerides only modestly during the 6 weeks of the investigations, BAIBA administration was able to fully prevent lipid accumulation in the liver during this period of time.

b)—Effect of BAIBA in Genetically Obese ob/ob Mice:

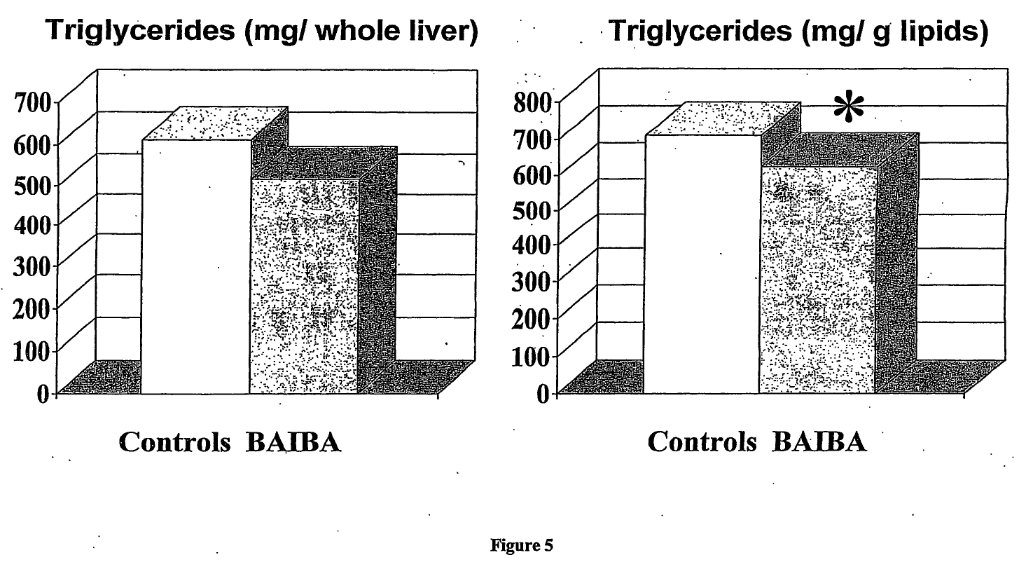

Liver triglycerides were assessed in ob/ob mice fed with a standard diet and receiving or not of 100 mg/kg/day of β-aminoisobutyric acid (BAIBA) in the drinking water during 6 weeks. Mice were 10 weeks old at the beginning of the experiment. These animals (7 controls and 10 treated with BAIBA) were different from those used for the determination of body fat by DEXA in the fed state (see above, paragraph I-d). At the end of the 6 weeks of treatment mice were submitted to a 48-hour period of fast At the end of this fasting period mice were killed for assessment of liver triglycerides. Obese ob/ob mice present a massive accumulation of triglycerides in the liver, and BAIBA was found to reduce hepatic triglyceride levels. Indeed, liver triglycerides were reduced by 16 and 12%, respectively when the values were expressed as mg/whole liver or as mg/gram of lipids. The results are submitted in FIG. 5 which shows that BAIBA afforded a significant reduction (p<0.01) of liver triglycerides from 709±18 to 625±14 mg/g of lipids. Although the diminution of hepatic triglycerides was moderate, these results suggest that BAIBA present some beneficial effects in ob/ob mice, a model of morbid obesity and massive hepatic steatosis.

c)—Effect of β-Aminoisobutyric Acid on Liver Histology in ob/ob Mice After 4 Months of Treatment:

Liver histology was first examined in ob/ob mice fed with standard diet receiving (11 mice) or not (10 mice) β-aminoisobutyric acid in the drinking water. In this experiment, mice were submitted to a 48-hr period of fast before killing and liver examination.

Livers of control ob/ob mice are characterized by massive steatosis. Indeed, in the control group, 8 out of 10 mice (80%) had almost all their hepatocytes (between 90 and 100%) engorged with fat. In the group treated with β-aminoisobutyric acid, only 3 mice out of 11 (27%) had between 90 and 100% of fat-laden hepatocytes.

In control ob/ob mice, perisinusoidal fibrosis, necrosis and inflammation were observed respectively in 60, 80 and 80% of the animals. In the treated group, however, these lesions were observed respectively in 45, 63 and 63% of the animals.

In this experiment, plasma alanine aminotransferase (i.e. an enzyme whose plasma level can increase as a result of liver injury) was not decreased in mice treated with β-aminoisobutyric acid.

In a second series of investigations, liver histology was studied in ob/ob mice treated for 4 months with β-aminoisobutyric acid, administered either at the usual dose (100 mg/kg/day) or at a higher dose (500 mg/kg/day). In this experiment, liver examination was performed in mice not submitted to a fasting period before sacrifice. The number of investigated animals were 6, 7 and 8, respectively in the control group and in the groups treated with 100 and 500 mg/kg/day of β-aminoisobutyric acid.

Livers of control ob/ob mice were characterized by massive steatosis (6 out of 6), perisinusoidal fibrosis (6 out of 6), portal or perivenular (centrilobular) fibrosis (6 out of 6), necrosis (6 out of 6) and inflammation (6 out of 6)(note that in this experiment necrosis and inflammation were scored separately). Thus, in control ob/ob mice, steatosis, perisinusoidal fibrosis, portal or perivenular fibrosis, necrosis and inflammation were observed in 100% of the animals.

Livers of ob/ob mice treated with 100 and 500 mg/kg/day of β-aminoisobutyric acid presented about the same extent of steatosis. However, when liver triglycerides were measured as described in the Materials and Methods, a slight trend toward lower triglycerides were observed. Indeed, liver triglycerides were 736±31, 721±16, 707±29 mg/g of lipids in control ob/ob mice and in ob/ob mice treated with 100 and 500 mg/kg/day of β-aminoisobutyric acid.

Contrasting with steatosis, other liver lesions were clearly less frequently observed in mice treated with β-aminoisobutyric acid. Indeed, perisinusoidal fibrosis was present in 29% of the treated mice, portal or perivenular fibrosis in 29%, necrosis in 43% and inflammation in 43% for mice treated with 100 mg/kg/day of β-aminoisobutyric acid) against 100% for the control ob/ob mice (see previously). In this experiment, plasma alanine aminotransferase (ALT) tended to be decreased in treated mice at the end of the treatment (i.e. after 4 months). Indeed, ALT was 311±42, 290±61 and 258±27 U/L, respectively in control mice and in mice treated with 100 and 500 mg/kg/day of β-aminoisobutyric acid.

Taken together, these data indicate that although the hepatic lesions defining steatohepatitis (i.e. fibrosis, necrosis and inflammation) are rather mild (except steatosis which is massive) in ob/ob mice, the β-aminoisobutyric acid can afford some beneficial effects in reducing the appearance of these lesions in a murine model of obesity, diabetes and steatosis.

### EXAMPLE III

### Beneficial Effects of BAIBA on Plasma Lipids Following a 6 Weeks Period of Treatment

a) Effect of BAIBA in Lean Mice Fed with a Standard Diet:

Plasma lipids were assessed in lean mice fed with a standard diet and receiving or not of 100 mg/kg/day of β-aminoisobutyric acid (BAIBA) in the drinking water during 6 weeks. There were six animals per group. At the end of the 6 weeks of treatment, mice were not fasted and samples of blood were then collected for the assessment of plasma triglycerides and total cholesterol. In this experiment, plasma triglycerides and total cholesterol were slightly decreased by BAIBA. Indeed, plasma triglycerides were 3.18±0.23 and 2.95±0.21 mmol/L, respectively in control and treated mice whereas plasma total cholesterol was 5.35±0.35 and 5.23±0.28 mmol/L, respectively in control and treated mice.

b)—Effect of BAIBA in Lean Mice Fed with a Western Diet:

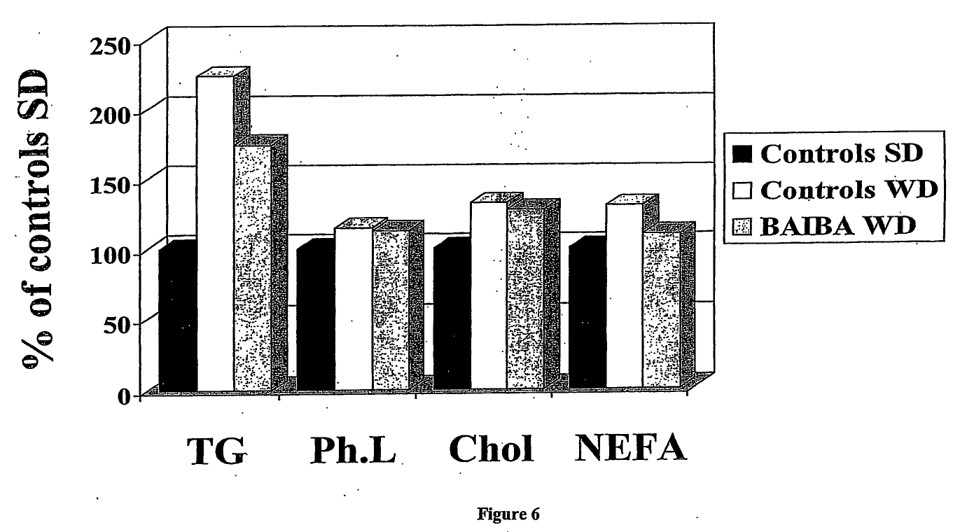

Plasma lipids were assessed in mice fed with the western diet (WD) and receiving or not of 100 mg/kg/day of β-aminoisobutyric acid (BAIBA) in the drinking water (8 mice in each group). For comparison, the experiment also included a group of mice (n=8) fed with a standard diet (SD). These animals were those used for the determination of body fat by DEXA and liver lipids (see above, paragraph I-c and II-a, respectively). After the last DEXA measurement (after 6 weeks of treatment), mice were allowed to recover from the anaesthesia for ca. 2 days and were then submitted to a 48-hour period of fast. At the end of this fasting period, a sample of blood was collected (from the retroorbital sinus) before DEXA measurements and plasma triglycerides, phospholipids, total cholesterol and non-esterified fatty acids (NEFA) were subsequently determined. After 6 weeks, the western diet was responsible for an increase of all plasma lipids, but the most affected lipids were triglycerides. The results are shown in FIG. 6.

Interestingly, BAIBA reduced by 22% plasma triglycerides in mice fed with the western diet, although the difference was not significant between control and treated mice (FIG. 6). Indeed, plasma triglycerides were 0.88±0.07, 1.96±0.45 et 1.54±0.13 mmol/L, respectively in control mice fed the standard diet (Controls-SD), control mice fed with the western diet (Controls-WD), and mice fed with the western diet and receiving BAIBA (BAIBA-WD). The western diet was also responsible for an increase in plasma NEFA and BAIBA tended to decrease by 16% NEFA in mice fed with this diet (FIG. 6). Indeed, plasma NEFA were 0.81±0.09, 1.05±0.08 and 0.88±0.06 mmol/L, respectively in the Controls-SD, Controls-WD and BAIBA-WD groups. Finally, BAIBA tended to decrease total cholesterol by 4% in mice fed with the western diet (from 6.98±0.64 to 6.71±0.27 mmol/L) (FIG. 6). All together, these results suggest that BAIBA is able to reduce plasma triglycerides when these lipids are abnormally increased by a western diet.

c)—Effect of BAIBA in Genetically Obese ob/ob Mice:

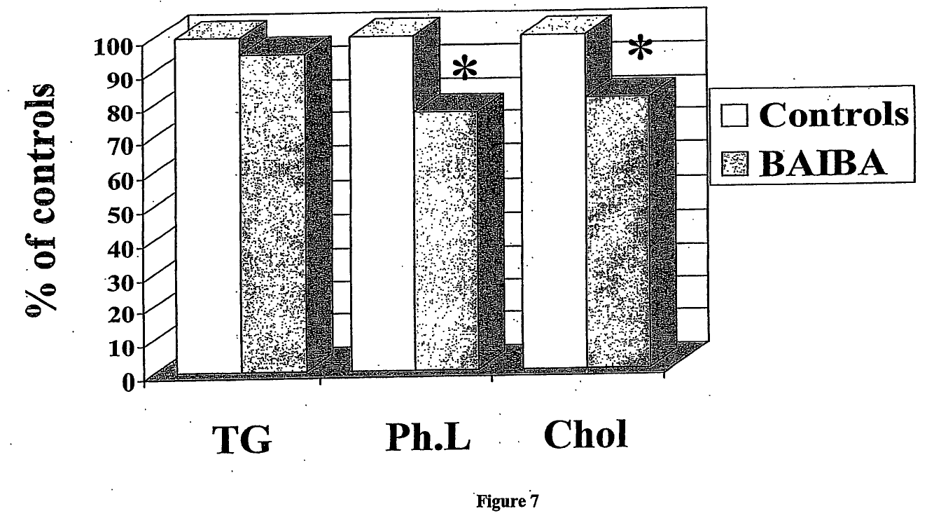

Plasma lipids were assessed in ob/ob mice fed with a standard diet and receiving or not β-aminoisobutyric acid (BAIBA) in the drinking water during 6 weeks. These animals (7 controls and 10 treated with BAIBA) were those used for the determination of hepatic lipids (see above, paragraph II-b). At the end of the 6 weeks of treatment, mice were submitted to a 48-hour period of fast. Samples of blood were then collected (from the retroorbital sinus) for the assessment of plasma triglycerides, phospholipids and total cholesterol and mice were subsequently killed for assessment of liver triglycerides (see above). In this experiment, BAIBA decreased by 5, 23 and 19%, respectively plasma triglycerides, phospholipids and total cholesterol. The results are submitted in FIG. 7. They show that:
plasma triglycerides were 1.39±0.22 and 1.32±0.14 mmol/L, respectively in control and treated ob/ob mice;
plasma phospholipids were significantly decreased by BAIBA from 5.27±0.32 to 4.07±0.26 mmol/L (p<0.01);
plasma total cholesterol was significantly decreased by BAIBA from 6.13±0.34 to 4.98±0.34 mmol/L (p<0.05).

In a second series of experiments, plasma lipids were assessed in ob/ob mice fed with a standard diet and receiving or not of 100 mg/kg/day of β-aminoisobutyric acid (BAIBA) in the drinking water during 6 weeks, but at the end of the experiment mice were fasted for only 15 hours (i.e. for an overnight fast). Samples of blood were then collected in controls mice (n=4) and in mice treated with BAIBA (n=5) for the assessment of plasma triglycerides, total cholesterol and HDL-cholesterol. In this experiment, BAIBA decreased plasma triglycerides and total cholesterol by 10 and 8%, respectively. Indeed, plasma triglycerides were 1.35±0.07 and 1.22±0.06 mmol/L, respectively in control and treated ob/ob mice, whereas plasma total cholesterol was 6.78±0.51 and 6.24±0.11 mmol/L, respectively in control and treated mice. Interestingly, there was a trend toward higher HDL-cholesterol in mice treated with BAIBA. Indeed, HDL-cholesterol was 4.73±0.31 and 4.92±0.24 mmol/L, respectively in control and treated mice.

Altogether, these results suggest that BAIBA has favourable effects on plasma lipids in ob/ob mice. However, it is noteworthy that ob/ob mice do not present marked hypertriglyceridemia (Lombardo Y. B., Hron W. T., Sobocinski K. A., Menahan L. A., 1983, Horm Metabol. Res. 16 : 37-42). Accordingly, plasma triglycerides were 1.35 and 1.39 mmol/L in ob/ob mice in the present study, whereas much higher levels (1.96 mmol/L) were found in Swiss mice fed with the western diet (see above). Thus, it remains possible that the beneficial effects of BAIBA on plasma lipids are more pronounced when lipid levels are above a certain threshold. In keeping with this notion, BAIBA reduced by 22% plasma triglycerides in mice fed with the western diet (FIG. 6). Moreover, we found that the beneficial effect of BAIBA on plasma lipids was less obvious in very young ob/ob mice (6 weeks at the beginning of the investigations, in contrast to 10-12 weeks for the above-described experiment [FIG. 7]) that present lower levels of plasma lipids. Indeed, we found that plasma triglycerides were unchanged (1.07.±0.06 and 1.07±0.03 mmol/L, respectively in the control [n=8] and treated [n=8] groups), whereas BAIBA only slightly reduced plasma phospholipids by 4% (from 4.51±0.09 et 4.35±0.11 mmol/L) and total cholesterol by 5% (from 5.24±0.11 to 4.96±0.15 mmol/L).

### EXAMPLE IV

### Beneficial Effects of BAIBA on Plasma HDL-Cholesterol and Triglycerides During a 4-Month Period of Treatment:

In this series of experiments, plasma lipids, and especially HDL-cholesterol were assessed in ob/ob mice during a 4-month period of treatment. Mice were fed with standard diet. Ob/ob mice were treated for 4 months with β-aminoisobutyric acid, administered either at the usual dose (100 mg/kg/day) or at a higher dose (500 mg/kg/day). After two months, measurements were done in the fed state, while at the end of the treatment (i.e. after 4 months) plasma parameters were assessed after a overnight fasting period (i.e. 15 hours). One mice in the control group died between the third and fourth month of treatment.

The number of mice at the end of the experiment was 6,7 and 8, respectively in the control group and the groups treated with 100 and 500 mg/kg/day of β-aminoisobutyric acid. It is noteworthy that the animals studied in this experiment are the same than those cholesterol. Indeed, HDL-cholesterol was 3.26±0.33, 3.45±0.31 and 3.64±0.31 mmol/L, respectively in control mice and mice treated with 100 and 500 mg/kg/day of β-aminoisobutyric acid. Plasma triglycerides tended to be decreased in treated mice. Triglycerides were indeed 1.37±0.13, 1.09±0.06 and 1.25±0.17 mmol/L, respectively in control mice and mice treated with 100 and 500 mg/kg/day of β-aminoisobutyric acid.

After 4 months (fasted state), it was again observed a trend toward an increase of HDL-cholesterol. Indeed, HDL-cholesterol was 3.89±0.49, 4.01±0.30 and 3.98±0.31 mmol/L, respectively in control mice and mice treated with 100 and 500 mg/kg/day of β-aminoisobutyric acid. Plasma triglycerides were 1.37±0.08, 1.34±0.08 and 1.26±0.07 mmol/L, respectively in control mice and mice treated with 100 and 500 mg/kg/day of β-aminoisobutyric acid.

Along with the data obtained with the 6-weeks treatment protocol, results regarding plasma cholesterol and notably HDL-cholesterol suggest that β-aminoisobutyric acid tends to balance blood level of cholesterol in favour of HDL-cholesterol, when administered over a 4-month period of time.

### EXAMPLE V

### Beneficial Effects of BAIBA on Glucose, Insulin, Adiponectin and Leptin:

a)—Effect of BAIBA in Lean Mice Fed with Standard Diet:

Glucose, insulin, adiponectine and leptin were assessed in lean mice fed with the standard diet and receiving (13 mice) or not (12 mice) 100 mg/kg/day of β-aminoisobutyric acid in the drinking water for 6 weeks.

At the end of the 6-weeks period of treatment, mice were submitted to a fasting period for 48 hours. Plasma glucose, insulin and adiponectin were not changed in treated mice, but a trend toward lower leptin levels was observed. Indeed, plasma leptin was 3.72±0.59 and 3.14±0.34 ng/ml, respectively in control mice (n=12) and mice treated with β-aminoisobutyric acid (n=13). Leptin is an adipokine mainly secreted by adipose tissue and its plasma levels in human and mice are highly correlated with the whole adipose tissue mass (Friedman J. M. and Hallas J. L., 1998, Nature 395: 763-770). Thus, data regarding plasma leptin are in keeping with the observation that β-aminoisobutyric acid limits body fat accumulation in lean mice.

In a second series of experiments plasma glucose was assessed in lean mice fed with the standard diet and receiving (6 mice) or not (6 mice) 100 mg/kg/day of β-aminoisobutyric acid in the drinking water for 6 weeks, but at the end of the experiment, mice were not fasted. In this experiment, plasma glucose was slightly decreased by BAIBA. Indeed, plasma glucose was 11.5±0.8 and 10.7±0.4 mmol/L, respectively in control and treated mice.

b)—Effect of BAIBA in Genetically Obese ob/ob Mice:

In obese ob/ob mice (10 to 12-weeks old) treated with 100 mg/kg/d of β-aminoisobutyric acid for 6 weeks and fasted for the last 48 hours, there was a trend toward lower plasma levels of glucose and insulin. Indeed, plasma glucose was 11.1±1.4 and 9.0±0.9 mmol/L, respectively in control ob/ob mice (n=6) and mice treated with β-aminoisobutyric acid (n=8). Plasma insulin was 37±19 and 24±6 μU/ml, respectively in control mice (n=4) and mice treated with β-aminoisobutyric acid (n=5). Interestingly, plasma levels of insulin tended to be also reduced in the fed state. Indeed, insulin was 83±13 and 64±7 μU/ml, respectively in control mice (n=5) and mice treated with β-aminoisobutyric acid (n=5).

c)—Effect of BAIBA in Very Young Genetically Obese ob/ob Mice:

In another experiment, very young ob/ob mice (5-6 weeks old) were treated with 100 mg/kg/d of β-aminoisobutyric acid for 6 weeks and fasted for the last 48 hours. Although plasma glucose was not decreased in treated mice, there was a clear trend toward lower insulin and higher adiponectin. Indeed, plasma insulin was 56±9 and 37±5 μU/ml (p=0.08), respectively in control mice (n=7) and mice treated with β-aminoisobutyric acid (n=7). Plasma adiponectin was 8.0±1.1 and 10.9±1.2 μg/ml (p=0.08), respectively in control mice (n=7) and mice treated with β-aminoisobutyric acid (n=7).

1. A method for the treatment and/or the prevention of diseases linked to the accumulation of triglycerides in tissues and blood comprising at least the step of administering to a human or non human animal in need thereof, as therapeutically active agent, an effective amount of β-aminoisobutyric acid, derivative, prodrug, metabolite or complex thereof.

2. A method for the treatment and/or the prevention of diseases linked to an imbalance of cholesterol in disfavour of HDL-cholesterol in tissues and blood comprising at least the step of administering to a human or non human animal in need thereof, as therapeutically active agent, an effective amount of β-aminoisobutyric acid, derivative, prodrug, metabolite or complex thereof.

3. A method of treatment for lowering the blood levels of triglycerides comprising at least the step of administering to a human or non human animal in need thereof, as therapeutically active agent, an effective amount of β-aminoisobutyric acid, derivative, prodrug, metabolite or complex thereof.

4. A method of treatment for balancing the blood and/or tissues level of total cholesterol in favour of HDL-cholesterol comprising at least the step of administering to a human or non human animal in need thereof, as therapeutically active agent, an effective amount of β-aminoisobutyric acid, derivative, prodrug, metabolite or complex thereof.

5. The method according to claim 3 for treating or preventing hypertension.

6. A method of treatment for lowering liver triglyceride levels comprising at least the step of administering to a human or non human animal in need thereof, as therapeutically active agent, an effective amount of β-aminoisobutyric acid, derivative, prodrug, metabolite or complex thereof.

7. The method according to claim 6 for treating or preventing hepatic steatosis and related liver diseases.

8. A method for the treatment or prevention of an obese condition, said method comprising at least the step of administering to a human or non human animal in need thereof, as therapeutically active agent, an effective amount of β-aminoisobutyric acid, derivative, prodrug, metabolite or complex thereof.

9. A method of treatment for the reduction or inhibition of the gain of body fat comprising at least the step of administering to a human or non human animal in need thereof, as therapeutically active agent, an effective amount of β-aminoisobutyric acid, derivative, prodrug, metabolite or complex thereof.

10. A method for alleviating resistance or restoring sensitivity to insulin comprising at least the step of administering to a human or non human animal in need thereof, as therapeutically active agent, an effective amount of β-aminoisobutyric acid, derivative, prodrug, metabolite or complex thereof.

11. A method according to claim 10, for lowering the blood levels of insulin and/or glucose.

12. A method according to claim 10, for increasing the blood levels of adiponectin.

13. A method according to claim 10 for treating or preventing type 2 diabetes and related cardiovascular diseases.

14. The method according to claim 1, wherein the β-aminoisobutyric acid derivative is an organic or inorganic salt, an ester or amide thereof.

15. The method according to claim 1, wherein β-aminoisobutyric acid is of configuration L or D or under a form of a mixture of L and D configurations.

16. The method according to claim 1, wherein the animal is a human.

17. The method according to claim 1, wherein the animal is an agricultural animal.

18. The method according to claim 1, wherein the animal is a domestic animal.

19. The method according to claim 1, wherein the animal is a laboratory animal.

20. A method for the treatment and/or prevention of diseases linked to an accumulation of triglycerides in tissues and blood, comprising:
providing a pharmaceutical composition comprising β-aminoisobutyric acid, derivative, prodrug, metabolite or complex thereof as therapeutically active agent, and
administering the pharmaceutical composition to a human or non human animal.

21. A method for the treatment and/or prevention of diseases linked to an imbalance of blood and/or tissues level of cholesterol in disfavour of HDL-cholesterol, comprising:
providing a pharmaceutical composition comprising β-aminoisobutyric acid, derivative, prodrug, metabolite or complex thereof as therapeutically active agent, and
administering the pharmaceutical composition to a human or non human animal.

22. The method according to claim 20, wherein the method is for the treatment and/or prevention of hypertension, angina pectoris, myocardial infarction and/or hyperlipemia.

23. The method according to claim 20, wherein the method is for the treatment and/or prevention of hepatic steatosis, steatohepatitis and/or diabetes.

24. The method according to claim 20, wherein the method is for the treatment and/or prevention of the syndrome X (i.e. metabolic syndrome).

25. A method for the treatment and/or prevention of disease linked to an accumulation of insulin or glucose and/or decrease of adiponectin, comprising:
providing a pharmaceutical composition comprising β-aminoisobutyric acid as therapeutically active agent, and
administering the pharmaceutical composition to a human or non human animal.

26. The method according to claim 25 wherein the method is for alleviating the resistance or restoring the sensitivity to insulin.

27. The method according to claim 25 wherein the method is for the prevention and/or treatment of diabetes type 2.

28. The method according to claim 20, wherein β-aminoisobutyric acid is of configuration L or D or under a form of a mixture of L and D configurations, or the β-aminoisobutyric acid is a β-aminoisobutyric acid derivative which is an organic or inorganic salt, an ester or amide thereof.

29. A pharmaceutical composition comprising as therapeutically active agent at least an effective amount of β-aminoisobutyric acid, derivative, prodrug, metabolite or complex thereof.

30. The pharmaceutical composition according to claim 29 for the treatment and/or prevention of hypertension, fatty liver and metabolic syndrome.

31. The pharmaceutical composition according to claim 29 for the treatment and/or prevention of an obese condition.

32. The pharmaceutical composition according to claim 29 for the treatment and/or prevention of diabetes type 2.

33. The pharmaceutical composition according to claim 29, wherein β-aminoisobutyric acid is of configuration L or D or under a form of a mixture of L and D configurations, or the β-aminoisobutyric acid is a β-aminoisobutyric acid derivative which is an organic or inorganic salt, an ester or amide thereof.

34. The pharmaceutical composition according to claim 29, comprising a pharmaceutically acceptable carrier or excipient.

35. A nutritional composition comprising as nutritional active agent an efficient amount of β-aminoisobutyric acid, derivative, prodrug, metabolite or complex thereof effective to reduce or to prevent an increase in the total body fat mass in human or non human animal.

36. The nutritional composition according to claim 35, wherein β-aminoisobutyric acid is of configuration L or D or under a form of a mixture of L and D configurations, or the β-aminoisobutyric acid is a β-aminoisobutyric acid derivative which is an organic or inorganic salt, an ester or amide thereof.

37. A method for producing reduction of the fat mass in a human or non-human animal in need thereof, comprising administering thereto an effective amount of nutritional composition according to claim 35.
